(19) United States
(12) Patent Application Publication
(10) Pub. No.: US 2005/0059049 A1
(43) Pub. Date: Mar. 17, 2005

(54) Hairpin-labeled probes and methods of use

(75) Inventors: Phillip T. Moen JR., Foxboro, MA (US); Jan Trnovsky, Saugus, MA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: One Cell Systems, Inc., Cambridge, MA (US)

(21) Appl. No.: 10/886,315

(22) Filed: Jul. 7, 2004

Related U.S. Application Data
(60) Provisional application No. 60/485,471, filed on Jul. 7, 2003.

(51) Int. Cl. C12Q 1/68; C07H 21/04

(52) U.S. Cl. 435/6; 536/24.3; 536/25.32

(57) ABSTRACT

The present invention provides nucleic acid hybridization probes having a target-binding region and a labeled hairpin structure at at least one end of the probe. The hairpin-labeled probes include oligonucleotides, dendrimers, and primer-extended nucleic acids. The probes can be used in disclosed methods for detection of target nucleic acids. In addition, the oligonucleotide probes can be used in disclosed methods for primer-extension, including, e.g., random priming and PCR amplification, to produce the primer-extended hairpin-labeled probes. Also disclosed are kits comprising the hairpin-labeled oligonucleotide and dendrimer probes. Further, the present invention provides biomolecules (e.g., peptides, polypeptides, carbohydrates, lipids, and the like) that are labeled via linkage to labeled hairpin structures.

### CROSS-REFERENCES TO RELATED APPLICATIONS

This application claims the benefit of U.S. Provisional Patent Application No. 60/485,471, filed Jul. 7, 2003, which is incorporated herein by reference in its entirety.

### BACKGROUND OF THE INVENTION

Nucleic acid hybridization is a powerful tool for detection of target nucleic acids. However, current detection probes and methods suffer from certain disadvantages that compromise the ability to detect low levels of target nucleic acids in various applications. For example, due to their small size, oligonucleotide probes cannot easily be labeled using chemical (e.g., platinum or psoralen compounds) or enzymatic methods (e.g., random primer labeling, polymerase chain reaction labeling, or nick translation labeling). Most commonly, oligonucleotide labeling is performed during synthesis or, alternatively, post-synthesis using 3′-end labeling, which involves the addition of a labeled nucleotide to the 3′end of the oligonucleotide. A single labeled nucleotide can be added by using a “chain terminating” nucleotide; alternatively, non-terminating nucleotides can be used, resulting in multiple nucleotides being added to form a “tail” (FIG. 1A). However, disadvantages of “tailing” include, for example, variability in the “tail” length from experiment to experiment, the small amount of label typically added (a majority of “tailed” oligonucleotides have only 1-2 labels added), and the ability to only label a small mass amount of oligonucleotide.

For synthesis labeling, the other common method, labeled nucleotides (e.g., phosphoramidite nucleotides) are incorporated into the oligonucleotide during chemical synthesis. Labels can be added to the 5′, 3′, or both ends of the oligonucleotide (FIG. 1B) (see, e.g., U.S. Pat. No. 5,082,830), or at base positions internal to the ODN (FIG. 1C). However, internal labeling is not favored, due to the detrimental impact on oligonucleotide hybrid stability to the target nucleic acid caused by the presence of bulky labeled molecules. Further, internal labeling is limited by the number of cognate nucleotides present in the sequence.

Some current oligonucleotide probes include nucleotide sequences that form hairpin structures. (See, e.g., U.S. Pat. Nos. 5,674,683; 5,808,036; 6,114,121.) However, these probes also suffer from similar disadvantages as described above in that, for example, internal nucleotides are labeled or the oligonucleotide is labeled at the 5′ end. Further, while other oligonucleotides having hairpin structures have been developed as capture probes (see, e.g., U.S. Pat. Nos. 5,770,365; 6,380,377), these structures have not been designed for use as detection probes with increased detection sensitivity.

Current hybridization probes and methods, therefore, limit nucleic acid detection capability, thereby limiting their effective use in various procedures, including diagnostic and analytical applications. For example, using current probes and methods, viral loads of, e.g., Human Immunodeficiency Virus (HIV), Ebstein-Barr Virus (EBV), or Cytomegalovirus (CMV)) are often not detectable in asymptomatic patients, thereby limiting the ability to identify early stages of disease or to assess the types of cells predominantly infected during latency periods. Rapid, sensitive methods for assessing viral infection, including more precise identification of viral reservoirs, are needed to optimize therapeutic intervention.

Therefore, there is a need in the art for oligonucleotide hybridization probes labeled for improved detection sensitivity The compositions and methods provided herein meet these and other needs.

### BRIEF SUMMARY OF THE INVENTION

The present invention provides a labeled oligonucleotide comprising (1) a single-stranded target-binding segment substantially complementary to a target nucleic acid and (2) a hairpin structure comprising a stem region and a loop region, in which two or more nucleotides within the hairpin structure have a detectable label. In certain embodiments, the labeled oligonucleotide also includes a linker between the target-binding segment and the hairpin structure. In various embodiments, hairpin nucleotide(s) having the detectable label are within the loop region, stem region, or both the loop and stem regions. In particular embodiments, at least five (5) nucleotides (e.g., nine (9) nucleotides) are detectably labeled. In addition, in certain embodiments of the present invention, the hairpin-labeled oligonucleotides can have, for example, up to 60, up to 100, or up to 150 nucleotides. In other embodiments, the loop region has 3-10 nucleotides and/or the stem region has 16-40 nucleotides. The detectably-labeled nucleotides can be adjacent or spaced at least two nucleotides apart (e.g., 2-6 nucleotides apart).

In some embodiments, the target-binding segment is a predetermined segment (i.e., designed according to a predetermined nucleic acid such as, for example, a viral nucleic acid (e.g., a HIV or EBV nucleic acid). In other embodiments the target-binding segment is a random segment or a degenerate segment.

In other embodiments, the detectable label is an indirect label such as, for example, biotin or a hapten (e.g., digoxigenin, dinitrophenol (DNP), biotin, and fluorescein). In yet other embodiments, the detectable label is a direct label such as, for example, a fluorophore (e.g., fluorescein, rhodamine, Texas Red, phycoerythrin, Cy3, and Cy5).

In still other embodiments, the present invention provides a labeled oligonucleotide comprising (1) a single-stranded target-binding segment substantially complementary to a target nucleic acid; (2) a first hairpin structure comprising a first stem region and a first loop region; and (3) a second hairpin structure comprising a second stem region and a second loop region; in which at least one nucleotide within the first hairpin structure and at least one nucleotide within the second hairpin structure have a detectable label, and in which the hairpin structures are linked to opposite ends of the target-binding segment. In certain embodiments, at least two nucleotides are detectably labeled within the first, second, or both hairpin structures.

In certain embodiments, the present invention also provides a dendrimer probe that includes (1) two or more labeled oligonucleotides comprising (a) a single-stranded target-binding segment substantially complementary to a target nucleic acid and (b) a hairpin structure comprising a stem region and a loop region, in which two or more nucleotides within the hairpin structure have a detectable label; and (2) a branching molecule linking the oligonucleotides.

In yet other embodiments, the present invention provides a labeled biomolecule that includes (1) an oligonucleotide that forms a hairpin structure comprising a stem region and a loop region, in which a plurality of nucleotides within the hairpin structure have a detectable label; and (2) a linker attaching the oligonucleotide and the biomolecule.

The present invention also provides a method for detecting a target nucleic acid in a sample. The method includes the following steps: (1) contacting the sample with an oligonucleotide probe, the oligonucleotide probe comprising (a) a single-stranded target-binding segment substantially complementary to the target nucleic acid; and (b) a hairpin structure comprising a stem region and a loop region, in which a plurality of nucleotides within the hairpin structure have a detectable label; (2) incubating the sample and the oligonucleotide probe under conditions sufficient to allow the target-binding segment to hybridize to the target nucleic acid; and (3) detecting the label on hybridized oligonucleotide probe to detect the target nucleic acid. In some embodiments, the method further includes removing non-hybridized oligonucleotide probe before detecting the label. In addition, in various embodiments, the oligonucleotide probe used in the detection method is any of the hairpin-labeled oligonucleotides as set forth above. In another embodiment, the probe used is the hairpin-labeled dendrimer probe as set forth above.

Further, in certain embodiments of the method in which the detectable label is an indirect label, the detection includes contacting the indirect label with a secondary label. For example, where the indirect label is biotin, the indirect label can be, e.g., streptavidin. Similarly, where the indirect label is a hapten, the secondary label can be, e.g., a labeled anti-hapten antibody.

In yet other embodiments of the detection method, the target nucleic acid is immobilized on a solid substrate. Alternatively, in other embodiments, the target nucleic acid is within a cell or tissue sample and the labeled oligonucleotide hybridizes to the target nucleic acid in situ. In certain embodiments, the detection of the label on hybridized oligonucleotide probe comprises a solution phase assay such as, for example, an assay that includes flow cytometry.

The present invention also provides a method for primer extension that includes contacting a target nucleic acid with an oligonucleotide primer under conditions whereby the target nucleic acid serves as a template for extension from the primer to produce an extended primer, the oligonucleotides primer comprising (1) a single-stranded target-binding segment substantially complementary to a target nucleic acid and (2) a hairpin structure comprising a stem region and a loop region, in which two or more nucleotides within the hairpin structure have a detectable label. In certain embodiments, the method further includes contacting the target nucleic acid with a second primer that comprises a priming segment substantially complementary to the extended primer, under conditions whereby the target nucleic acid serves as a template for amplification from the oligonucleotide primer and the second primer to produce an amplification product. The second primer can, for example, include a second hairpin structure comprising a second stem region and a second loop region, in which at least one nucleotide within the second hairpin structure has the detectable label. In certain embodiments comprising the use of two hairpin-labeled primers, at least one nucleotide in the hairpin structure of each of the first and second primers is detectably labeled.

In some embodiments of the primer extension method, the amplification is performed in the presence of unlabeled free nucleotides. In other embodiments, the target-binding segment is random (for example, random segments having, e.g., 3-10 nucleotides).

The present invention also provides a kit for detection of a target nucleic acid, the kit comprising at least one first container providing either (1) a labeled oligonucleotide comprising (a) a single-stranded target-binding segment substantially complementary to a target nucleic acid and (b) a hairpin structure comprising a stem region and a loop region, in which two or more nucleotides within the hairpin structure have a detectable label; or (2) a dendrimer probe comprising (a) two or more labeled oligonucleotides as set forth in (1) and (b) a branching molecule linking the oligonucleotides. In certain embodiments, the detectable label is an indirect label and the kit further includes at least one second container providing a secondary agent for detecting the indirect label.

The present invention further provides a kit for primer extension of an oligonucleotide primer, the kit comprising at least one first container providing a labeled oligonucleotide primer comprising (a) a single-stranded target-binding segment substantially complementary to a target nucleic acid and (b) a hairpin structure comprising a stem region and a loop region, in which two or more nucleotides within the hairpin structure have a detectable label, and in which the hairpin structure is located 5′ to the target-binding segment. In certain embodiments, the kit further comprises at least one second container providing a second primer, the second primer comprising a priming segment substantially complementary to an extended primer produced under conditions whereby the target nucleic acid serves as a template for extension from the labeled oligonucleotide primer. In yet other embodiments, the kit also includes at least one third container providing labeled or unlabeled free nucleotides, at least one fourth container providing a polymerization agent, and at least one fifth container providing a buffer suitable for primer extension.

### BRIEF DESCRIPTION OF THE DRAWINGS

FIGS. 1A and 1B depict examples of different types of oligonucleotide probes: (A) 3′-biotin-tailed oligonucleotide; (B) 3′,5′-biotinylated oligonucleotide; and (C) internally biotinylated oligonucleotide.

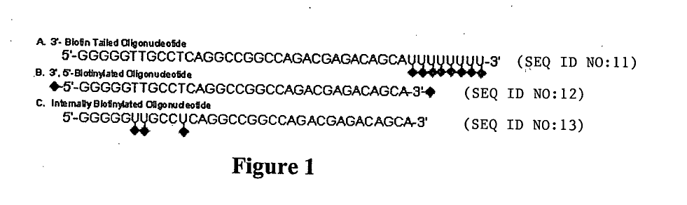

FIG. 2 depicts an example of a hairpin-labeled oligonucleotide, showing a sequence complementarity to a target region, a short linker, and a labeled hairpin with stem and loop regions.

FIGS. 3A and 3B depict a schematic representation of generation of labeled probes by PCR (A) and random priming (B) using hairpin-labeled oligonucleotide primers.

FIGS. 4A and 4B depict detection of nascent ribosomal RNA using hairpin-labeled oligonucleotides. Fluorescence in situ hybridization (FISH) was performed using hairpin-labeled or conventional 3′,5′-biotinylated oligonucleotide probes targeting sequences within Intervening Transcribed Sequence-1 (ITS-1) nascent ribosomal RNA. After hybridization, bound probe was detected using Cy3-conjugated streptavidin, and samples were digitally imaged identically. (A) Detection of ITS-1 RNA using conventional biotin labeling. Hybridization signal is specific for nucleoli only, consistent with the expected localization of nascent ribosomal RNA. (B) Detection of ITS-1 using hairpin-labeled oligonucleotide probe shows the same labeling specificity as in (A), but exhibits a noticeably stronger hybridization signal.

FIG. 5 depicts flow cytometry comparison of hybridization signal intensities using different oligonucleotide probes. Synthetic oligonucleotide probes targeting sequences within Intervening Transcribed Sequence-1 (ITS-1) of nascent ribosomal RNA were labeled using conventional 3′,5′-biotinylation (C) or hairpin labeling (D). After solution-phase hybridization, signal intensities were measured using flow cytometry, and resultant histogram tracings plotted. For comparison, samples containing no probe (A) or an irrelevant probe (B) also were analyzed. Hairpin labeling (green) resulted in an approximate 50% increase in signal intensity over conventional labeling (C).

FIGS. 6A-6F depict a diagram and sequence of representative oligonucleotide probes for nascent ribosomal RNA and Epstein Barr Virus EBER-1 RNA. (A). ITS-1 nascent ribosomal RNA conventional probe. (B). ITS-1 hairpin-labeled probe. (C). EBER-1 RNA conventional probe. (D). EBER-1 RNA hairpin-labeled probe. (E). Biotinylated hairpin labeling stem and loop structure and sequence. (*) denotes biotin label. Underlined sequences in panels (B) and (D) denote sequence complementary to target region.

FIGS. 7A-7D depict four different labeled hairpin structures showing different distributions of biotins in the labeling scheme.

### DETAILED DESCRIPTION OF THE INVENTION

I. Definitions

Unless defined otherwise, all technical and scientific terms used herein have the same meaning as commonly understood by one of ordinary skill in the art to which this invention belongs. Although any methods and materials similar to those described herein can be used in the practice or testing of the present invention, only exemplary methods and materials are described. For purposes of the present invention, the following terms are defined below.

The terms “a,” “an,” and “the” are not limiting and shall include plural referents, unless the context clearly indicates otherwise.

The term “nucleotide”, in addition to referring to the naturally occurring ribonucleotide or deoxyribonucleotide monomers, shall herein be understood to refer to related structural variants thereof, including derivatives and analogs, that are functionally equivalent with respect to the particular context in which the nucleotide is being used (e.g., formation of hairpin structure, hybridization to complementary base, or linkage of two non-adjacent nucleic acid segments), unless the context clearly indicates otherwise.

The term “nucleic acid” and “polynucleotide” are synonymous and refer to a polymer having multiple nucleotide monomers. A nucleic acid can be single- or double-stranded, and can be DNA (cDNA or genomic), RNA, synthetic forms, and mixed polymers, and can also be chemically or biochemically modified or can contain non-natural or derivatized nucleotide bases. Such modifications include, for example, methylation, substitution of one or more of the naturally occurring nucleotides with an analog, internucleotide modifications such as uncharged linkages (e.g., methyl phosphonates, phosphotriesters, phosphoamidates, carbamates, and the like), charged linkages (e.g., phosphorothioates, phosphorodithioates, and the like), pendent moieties (e.g., polypeptides), intercalators (e.g., acridine, psoralen, and the like), chelators, alkylators, and modified linkages (e.g., alpha anomeric nucleic acids, and the like). Also included are synthetic molecules that mimic polynucleotides in their ability to bind to a designated sequence via hydrogen bonding and other chemical interactions. Typically, the nucleotide monomers are linked via phosphodiester bonds, although synthetic forms of nucleic acids can comprise other linkages (e.g., peptide nucleic acids as described in Nielsen et al., supra, Science 254, 1497-1500, 1991). “Nucleic acid” or “polynucleotide” do not refer to any particular length of polymer and can, therefore, be of substantially any length, typically from about six (6) nucleotides to about 10⁹ nucleotides or larger. In the case of a double-stranded polymer, “nucleic acid” or “polynucleotide” can refer to either or both strands.

The term “oligonucleotide” refers to a subset of polynucleotide of from about 6 to about 175 nucleotides or more in length. Typical oligonucleotides are up to about 100 nucleotides in length. Oligonucleotides can be synthesized using known methods (e.g., using an automated oligonucleotide synthesizer such as, for example, those manufactured by Applied BioSystems (Foster City, Calif.)).

The term “target nucleic acid” means a nucleic acid which is to be detected or which is to serve as a template for priming (e.g., PCR or random priming). A target nucleic acid can be single-stranded or double-stranded, although, for uses described herein, double-stranded targets are generally made single-stranded using known methods. Target nucleic acids can include, e.g., prokaryotic, eukaryotic, or viral polynucleotides from essentially any natural source having the nucleic acids (e.g., cells, tissues, or biological fluids).

An “oligonucleotide probe” is defined as an oligonucleotide capable of binding to a target nucleic acid of substantial complementarity through one or more types of chemical bonds, usually through complementary base pairing, usually through hydrogen bond formation. A probe can include natural (i.e., A, G, C, or T) or modified bases (e.g., 7-deazaguanosine, or inosine). In addition, the bases in a probe can be joined by a linkage other than a phosphodiester bond, so long as it does not interfere with hybridization. For example, oligonucleotide probes can be peptide nucleic acids in which the constituent bases are joined by peptide bonds rather than phosphodiester linkages.

A “labeled oligonucleotide” is an oligonucleotide that is bound, either covalently, through a linker, or through ionic, van der Waals or hydrogen bonds to a label such that the presence of the probe can be detected by detecting the presence of the label bound to the probe.

An “indirect label” is a specifically bindable molecule (a “ligand”) that is detected using a labeled secondary agent (a “ligand binding partner”) that specifically binds to the indirect label. Conversely, a “direct label” is detected without a ligand binding partner interaction. For indirect labels, the secondary agent typically has a direct label, or, alternatively, the secondary agent can also be labeled indirectly. Typical “direct labels” include, for example, fluorophores (e.g., fluorescein, rhodamine, or phthalocyanine dyes), chromophores (e.g., phycobiliproteins), luminescers (e.g., chemiluminescers and bioluminescers), lanthanide chelates (e.g., complexes of Eu³⁺ or Tb³⁺), enzymes (e.g., alkaline phosphatase), cofactors, and residues comprising radioisotopes such as, e.g., ³H, ³⁵S, ³²P, ¹²⁵I, and ¹⁴C. Typical indirect labels include, e.g., haptens, biotin, or other specifically bindable ligands.

A “hapten” means an isolated epitope, i.e., a molecule having an antigenic determinant. Examples of haptens include dinitrophenol (DNP), digoxigenin, biotin, and fluorescein. As an indirect label, a hapten is typically detected using an anti-hapten antibody as the ligand binding partner. However, a hapten can also be detected using an alternative ligand binding partner (e.g., in the case of biotin, anti-biotin antibodies or streptavidin, for example, can be used as the ligand-binding partner). Further, in certain embodiments, a hapten can also be detected directly (e.g., in the case of fluorescein, an anti-fluorescein antibody or direct detection of fluorescence can be used).

“Similarity,” in the context of two nucleic acids, mean that the two nucleic acids have similar nucleotide sequences when compared and aligned for maximum correspondence (as measured by visual inspection or using a sequence comparison algorithm such as, e.g., PILEUP or BLAST). For example, two nucleic acids are similar if they share at least 40%, at least 50%, at least 60%, at least 70%, at least 80%, at least 90%, or at least 95% nucleotide identity when compared and aligned for maximum correspondence. Two nucleic acids are “substantially similar” or “substantially identical” if they share at least 60%, typically at least 80%, and more typically at least 90%, at least 95%, or at least 99% nucleotide identity.

A “predetermined nucleic acid” refers to a nucleic acid that is used to design the target binding region of the oligonucleotide probe (i.e., for substantial complementarity). For example, the target nucleic acid or a nucleic acid with similarity to the target can be predetermined. A target-binding region that is designed according to a predetermined nucleic acid is herein a “predetermined target-binding region” or “predetermined segment” of the oligonucleotide probe.

In the context of an oligonucleotide probe, “random segment” and “random target-binding region” refer to a target binding region having randomly ordered nucleotides or derivatives thereof, e.g., there is an equal probability of any of the four bases occupying any the positions within the target binding region.

A “degenerate target-binding region” or “degenerate segment” refers to a target-binding region that is designed according to a predetermined peptide or polypeptide based on the degeneracy of the genetic code. A degenerate target-binding region, therefore, has both fixed and degenerate nucleotide positions. A “degenerate” nucleotide position has an equal probability of being occupied by any of two, three, or four bases depending on the corresponding amino acid and the codon position.

The term “sample” generally refers to a material of biological origin. Samples can include, e.g., tissues; cells; plasma; serum; spinal fluid; lymph fluid; tears; saliva; blood cells; hair; tumors; organs; the external sections of the skin, respiratory, intestinal, and genitourinary tracts. Samples can also include in vitro cell culture constituents of the above. Samples can be purified or semi-purified to remove certain constituents (e.g., non-polynucleotide or other non-target constituents).

“Substantially complementary” means that a nucleic acid strand is capable of hybridizing to a target nucleic acid strand. “Hybridization” means sufficient hydrogen bonding, which can be, e.g., Watson-Crick, Hoogsteen, or reversed Hoogsteen hydrogen bonding, between complementary nucleoside or nucleotide bases such that stable and specific binding occurs between the nucleic acid strands. Hybridization capability is determined according to stringent conditions, including suitable buffer concentrations and temperatures, that allow specific hybridization to a target nucleic acid having a region of full or partial complementarity. Thus, not all nucleotides of the nucleic acid need be complementary. Further, a nucleic acid strand is “substantially complementary” when it hybridizes to all, part, or an overlapping region of the target nucleic acid. Qualitative and quantitative considerations for establishing stringent hybridization conditions for the design of oligonucleotides according to the present invention are known in the art. (See, e.g., Ausubel et al., Short Protocols in Molecular Biology (4th ed., John Wiley & Sons 1999); Sambrook et al, Molecular Cloning: A Laboratory Manual (3d ed., Cold Spring Harbor Laboratory Press 2001); Nucleic Acid Hybridisation: A Practical Approach (B. D. Hames & S. J. Higgins eds., IRL Press 1985).) Stringent hybridization conditions can include, for example, 6×NaCl/sodium citrate (SSC) at about 45° C. for a hybridization step, followed by a wash of 2×SSC at 50° C.; or, alternatively, e.g., hybridization at 42° C. in 5×SSC, 20 mM NaPO4, pH 6.8, 50% formamide, followed by a wash of 0.2×SSC at 42° C. Typically, two nucleic acid regions are substantially complementary when, e.g., at least 90% of the respective bases are complementary, more typically when at least 95% and preferably when 100% of the respective bases are complementary.

“Tₘ” is defined as the temperature (under defined ionic strength and pH) at which 50% of the target sequence hybridizes to a perfectly matched probe.

The term “primer” refers to a polynucleotide capable of acting as a point of initiation of template-directed nucleic acid synthesis under appropriate conditions (i.e., in the presence of four different nucleoside triphosphates and an agent for polymerization, such as, DNA or RNA polymerase or reverse transcriptase) in an appropriate buffer and at a suitable temperature. Primers, therefore, include a target-binding region that hybridizes to a target nucleic acid (the template). Primers are typically an oligonucleotide and are single-stranded, although, a primer can refer to a polynucleotide having a double-stranded segment (e.g., an oligonucleotide having a single-stranded and a hairpin region, as described infra). The appropriate length of the target-binding region for a primer depends on the intended use of the primer but typically ranges from 6 to 40 nucleotides. Short primer molecules generally require cooler temperatures to form sufficiently stable hybrid complexes with the template. A primer need not reflect the exact sequence of the template but must be sufficiently complementary to hybridize with a template. The term primer site refers to the area of the target nucleic acid to which a primer hybridizes. The term primer pair means a set of primers including a 5′ upstream primer that hybridizes with the 5′ end of the nucleic acid sequence to be amplified and a 3′ downstream primer that hybridizes with the complement of the 3′ end of the sequence to be amplified.

The term “hairpin structure” refers to a nucleic acid having a double-stranded “stem” region and a single-stranded “loop” region, in which the stem region and loop region are formed from a single strand of nucleic acid having (1) two regions that are mutually, substantially complementary so as to form the double-stranded segment comprising the stem (i.e., via complementary base pairing) and (2) interposed between the two mutually complementary regions, a third region that forms the loop. Hairpin structures are described in, for example, Varani, Annu. Rev. Biophys. Biomol. Struct. 24:379-404, 1995.

The term “adjacent”, in reference to two segments of a nucleic acid, means that the segments are non-overlapping and not separated by an intervening segment.

A “linker,” in the context of attachment of two non-adjacent and non-overlapping segments of nucleic acid, means a molecule (monomeric or polymeric) that is interposed between and adjacent to the non-adjacent segments. The linker can be a nucleotide linker (i.e., a segment of the nucleic acid that is between and adjacent to the non-adjacent segments) or a non-nucleotide linker.

The term “solution phase assay” refers to any assay in which the a target nucleic acid is detected while in solution or in suspension (e.g., where the probe is detected while hybridized to the target nucleic acid in a suspension cell in situ using, for example, flow cytometry analysis).

“Isolated nucleic acid” refers to a nucleic acid removed from its original environment (e.g., the natural environment if it is naturally occurring). For example, a naturally occurring nucleic acid in a living animal is not isolated, but the same nucleic acid, separated from some or all of the co-existing materials in the natural system, is isolated. Such nucleic acids can be part of a vector and/or such nucleic acids can be part of a composition, and still be isolated in that such vector or composition is not part of its natural environment.

II. Hairpin-Labeled Probes

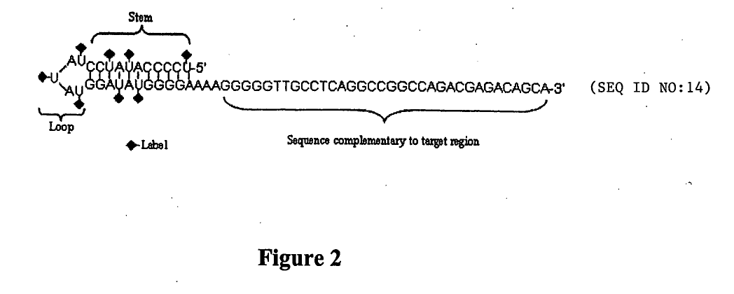

The present invention provides novel hybridization probes for detection of target nucleic acid sequences. The hybridization probe includes a synthetically derived oligonucleotide having one or both ends capable of forming a thermodynamically stable hairpin structure. Within the hairpin structure(s) are two or more nucleotides linked to a detectable molecule (see, e.g., FIG. 2). Labeling of multiple nucleotides with the hairpin structure(s) of each oligonucleotide probe increases the sensitivity of detection while maintaining stability of the probe-target hybrid, thereby allowing for detection of low levels of target nucleic acids. Also provided are dendrimeric probes having a plurality of labeled hairpin oligonucleotide branches. The hairpin-labeled oligonucleotides or dendrimers can be used for detection of nucleic acids in a variety of formats, including, e.g., in situ hybridization and tissue arrays, as well as for preparation of labeled nucleic acids by primer extension methods using the target nucleic acid as a template.

The oligonucleotides can be synthesized using known methods (see, e.g., Glick and Pasternak, Molecular Biotechnology: Principles and Applications of Recombinant DNA (ASM Press 1998)). Solution or solid-phase techniques can be used. Synthesis procedures are typically automated and can include, for example, phosphoramidite, phosphite triester, H-phosphate, or phosphotriester methods. The oligonucleotides are typically at least 25 or at least 30 nucleotides in length and can consist of up to 100, 125, 150, 175, or even more nucleotides. Typically, the labeled oligonucleotides have up to 90 or up to 80 nucleotides, and more typically up to 60 or up to 50 nucleotides. The synthesis method used can depend on the desired length of the oligonucleotide.

The hairpin region typically includes from about 18 to about 50 nucleotides, more typically from about 25 to about 40 nucleotides. Formation of the hairpin structure is accomplished via design of two subregions having substantial anti-parallel complementarity, i.e., sufficient complementarity running in opposite directions to specifically hybridize under stringent conditions, thereby forming a stem region. Between the substantially mutually complementary regions is interposed a non-complementary sequence, which does not hybridize intramolecularly and therefore has as a single-stranded loop configuration upon formation of the double-stranded stem region. The number and base composition of nucleotides in the loop region are selected to allow the mutually complementary subregions of the stem to hybridize. For example, the loop region is designed to avoid substantial complementarity with either strand of the stem region and typically has from 3 to 20 nucleotides, more typically from 3 to 10 nucleotides, and most typically from 5 to 8 nucleotides. As indicated supra, the number and base composition of nucleotides in the stem region are selected for substantial complementarity of two regions running in opposite directions such that a double-stranded hybrid of a desired relative stability is formed under stringent hybridization conditions. Typically, the stem region typically has from 14 to 46 nucleotides (i.e., 7-23 nucleotides for each strand of the stem), more typically from 16 to 40 nucleotides (i.e., 8-20 nucleotides for each strand of the stem), and most typically from 20 to 32 nucleotides (i.e., 10-16 nucleotides for each strand of the stem).

The target-binding region is non-overlapping with the hairpin region and is typically at least 3 or at least 6 nucleotides, more typically at least 10 nucleotides, even more typically at least at least 14 or at least 17 nucleotides, and still more typically at least 25 or at least 30 nucleotides in length. Target-binding regions having complementary sequences over stretches greater than 20 bases in length are generally preferred. The length and base composition of the target-binding region is generally selected to not interfere with formation of the attached hairpin structure and to not in itself form a stem-loop structure with equal or greater thermal stability that the labeled stem-loop structure under hybridization conditions appropriate for specific detection of the target nucleic acid.

The nucleotide sequence of the target-binding region can be, e.g., predetermined, random, or degenerate. For example, the predetermined target binding region can be designed to have substantial complementarity with a predetermined polynucleotide such as, e.g., a nucleic acid associated with an infectious agent (e.g., viral nucleic acids such as, for example, HIV or EBV nucleic acids). In the case of a random target-binding region, the probe having a random segment can be, e.g., from a random library of hairpin-labeled probes. For example, a library of labeled oligonucleotides having random target-binding regions can include those having target-binding regions that represent all possible sequences of length N (where N is a positive integer), or a subset of all possible sequences. In certain embodiments, the random target binding segment is 3-10 nucleotides in length. Similarly, for a degenerate target-binding region, the probes can be, e.g., from a library of hairpin-labeled oligonucleotides having target-binding regions that represent all possible coding sequences for a given peptide or polypeptide.

In certain embodiments of the invention, the labeled oligonucleotide can further include a linker interposed between and adjacent to the hairpin and target-binding regions. The linker is typically one or more nucleotides, including non-natural or derivatized nucleotides. The number and composition of bases for nucleotide linkers are selected to not interfere with formation of the hairpin structure or hybridization of the target-binding region to its target. Nucleotide linkers are typically from 1-20 nucleotides in length, more typically from 1-10 and even more typically from 1-5 nucleotides in length. Alternatively, non-nucleotide linkers of various lengths can be used. Suitable non-nucleotide linkers are known in the art and include, for example, Spacer Phosphoramidites C3, 9, and 18 (Glenn Research, #10-1913, 10-1909, or 10-1918, respectively).

The oligonucleotide is labeled internally within the hairpin structure, with two or more nucleotides within the stem-loop region linked to a detectable label. Labeling within the hairpin structure allows the introduction of multiple labels into the oligonucleotide without involving nucleotides that take part in hybrid formation with the target nucleic acid. In certain embodiments, at least 5 or at least 8 nucleotides within the hairpin structure are labeled; in one exemplary embodiment, the number of nucleotides having the detectable label is 9. Labeled nucleotides can be adjacent or non-adjacent. Typically, the labeled nucleotides are non-adjacent and are spaced throughout the hairpin structure. In the case of an indirect label, for example, the degree of spacing of the labels is designed to minimize steric hindrance of the ligand-ligand binding partner interaction of label with a secondary agent (e.g., biotin and streptavidin). In typical embodiments, the labeled nucleotides are spaced at least 2, at least 4, at least 6, or at least 8 nucleotides apart. In addition, spacing of labeled nucleotides can vary with a hairpin structure. In certain embodiments, the spacing varies from 2 or 3 nucleotides to 4, 6, 8, or more nucleotides apart. Further, in yet other embodiments, at least one nucleotide in the loop region, at least one nucleotide in the stem region, or at least one nucleotide in each of the loop and stem regions are labeled.

The detectable label can be direct or indirect. Labels suitable for use according to the present invention are known in the art and generally include any molecule that, by its chemical nature and whether by direct or indirect means, provides an identifiable signal allowing detection of the probe. Preferred direct labels include fluorophores such as, for example, fluorescein, rhodamine, Texas Red, phycoerythrin, and phthalocyanine dyes (e.g., Cy3 or Cy5). Other direct labels can include, for example, radionuclides and enzymes such as, e.g., alkaline phosphatase, horseradish peroxidase, or β-galactosidase. Alternatively, indirect labels can be used. For example, the indirect label can be biotin, which can be detected using, for example, labeled streptavidin (e.g., streptavidin conjugated to fluorescein). In other embodiments, the indirect label is a hapten which is detected using an anti-hapten antibody. Typical haptens suitable for use according to the present invention include, e.g., biotin, digoxigenin, dinitrophenol (DNP), and fluorescein.

The detectable label can be incorporated into the hairpin structure using known methods. Typically, the label is incorporated into the oligonucleotide during chemical synthesis using, e.g., labeled nucleotides or derivatives thereof such as labeled phosphoramidite nucleotides. For example, biotin phosphoramidites or phosphoramidites linked to flourescein dyes (e.g., 6-FAM™, HEX™, or TET™) can be used. Alternatively, labels can be attached through a linker moiety on the nucleotide or derivative thereof. For example, nucleotide derivatives (e.g., phosphoramidites) having linker moieties for attachment of labels can be used during synthesis, followed by a labeling reaction that imparts the label to the linker moiety. Alternatively, non-nucleotide monomers having a linker moiety for attachment of a label can be incorporated into the oligonucleotide during synthesis (for a description of non-nucleotide linking reagents for nucleotide probes, see, e.g., U.S. Pat. No. 5,585,481). Linker moieties can be protected or non-protected, and detectable labels can be attached prior or subsequent to polymer synthesis (e.g., following deprotection of the linker moiety). Further, the linker moiety can be designed for linkage to any of a variety of chemical structures, including, for example, biomolecules such as, e.g., polypeptides, peptides, carbohydrates, lipids, and the like. The biomolecule or other chemical structure for attachment can, for example, be a direct or indirect label (e.g., a fluorophore, enzyme, or a molecule having specific binding properties which allow for it use as an indirect label according to the methods provided herein such as, e.g., a hapten, biotin, or another ligand having specificity for a particular receptor or other ligand-binding partner). Alternatively, the chemical structure attached to the linker moiety can itself be detectably labeled. For example, a peptide labeled with, e.g., a fluorophore or biotin can be attached to the linker moiety.

In general, the site of label or linker attachment is not limited to any specific position. For example, a label can be attached to a nucleotide or derivative thereof at any position that does not interfere with detection or hairpin structure formation as desired. The base moieties of useful labeling reagents can include those that are naturally occurring or modified in a manner that does not interfere with the purpose to which they are put. Modified bases include, for example, 7-deaza A and G, 7-deaza-8-aza A and G, and other heterocyclic moieties.

In the case of fluorescent labels, fluorophores are not to be limited to single species organic molecules, but include inorganic molecules, multi-molecular mixtures of organic and/or inorganic molecules, crystals, heteropolymers, and the like. For example, CdSe-CdS core-shell nanocrystals enclosed in a silica shell can be easily derivatized for coupling to a biological molecule (Bruchez et al., Science, 281:2013-2016, 1998). Similarly, highly fluorescent quantum dots (zinc sulfide-capped cadmium selenide) have been covalently coupled to biomolecules for use in ultrasensitive biological detection (Warren and Nie, Science, 281: 2016-2018, 1998).

In certain embodiments of the invention, both ends of the oligonucleotide have hairpin structures with at least one nucleotide in each hairpin structure having the detectable label. In preferred embodiments, two or more nucleotides in each hairpin are labeled, more preferably at least five and even more preferably at least eight nucleotides in each hairpin. In certain embodiments, the terminal nucleotides (5′ and 3′) are not detectable labeled.

Dendrimer Probes

In another aspect, the hybridization probe is a dendrimer. The term “dendrimer” refers to branched macromolecules having polymeric “arms” that emanate from a core molecule. Thus, the dendrimeric hybridization probes of the present invention have two or more hairpin-labeled oligonucleotide arms linked to a central branching molecule. Methods of making “oligonucleotide dendrimers” are generally known in the art. (See, e.g., U.S. Pat. No. 6,455,071; U.S. Pat. No. 6,274,723; Azhayeva et al., Nucleic Acids Res. 23:1170-1176, 1995; Horn and Urdea, Nucleic Acids Res. 17:6959-6967, 1989.)

The “branching molecule” can be monomeric or polymeric, and linkage to the branching molecule can be via covalent or non-covalent interactions. Thus, dendrimers according to the present invention can include, for example, a plurality of oligonucleotides linked covalently to a branching molecule such as, e.g., a nucleoside derivative (such as described in, e.g., Azhayeva et al., supra; Horn and Urdea, supra) or a phosphoramidite synthon (see, e.g., Shchepinov et al., Nucleic Acids Res. 25:4447-4454, 1997). In other embodiments, the oligonucleotides are linked non-covalently to the branching molecule such as, for example, a nucleic acid polymer by, e.g., hybridization of substantially complementary regions. For example, the branching molecule can be a dimer of two partially single-stranded nucleic acids, linked at an internal region by complementary base pairing and having four single-stranded regions available for linkage to a hairpin-labeled oligonucleotide (see, e.g., U.S. Pat. No. 6,274,723).

Typically, the hairpin structure of each oligonucleotide branch has at least one detectable label. In preferred embodiments, each hairpin structure has two or more labels, more preferably at least five and even more preferably at least eight nucleotides in each hairpin.

Hairpin-Labeled Biomolecules

In another aspect, the present invention provides a labeled biomolecule that includes (1) a labeled oligonucleotide that forms a hairpin structure comprising a stem region and a loop region, with the hairpin structure having two or more nucleotides linked to a detectable molecule, and (2) a linker attaching the oligonucleotide and the biomolecule. The labeled hairpin structures that are linked to the biomolecules are essentially as described supra. “Biomolecules” refers to classes of molecules that exist in and/or can be produced in living systems as well as structures derived from such molecules. Suitable biomolecules typically include, for example, peptides, polypeptides, saccharides, fatty acids, steroids, purines, pyrimidines, and derivatives, structural analogs, or combinations thereof. In typical embodiments, the labeled biomolecule is a non-nucleic acid biomolecule such as, e.g., a peptide, polypeptide, carbohydrate, or lipid.

In addition, in typical embodiments, the biomolecule is capable of specifically interacting with a binding partner (a “target molecule”) through non-covalent interactions such as, for example, through hydrogen bonds, ionic bonds, van der Waals attractions, or hydrophobic interactions. Thus, the labeled biomolecules are useful, for example, in the detection of a particular target molecule that has a specific binding affinity for the biomolecule. “Target molecules” can include, for example, soluble protein, cytokines, chemokines, cell membrane proteins, cellular receptors, glycoproteins, or other macromolecules. A target molecule can be localized, for example, within the cytosol, on the surface of a cell, on the surface of an isolated subcellular organelle, in solution, or in extracellular spaces. For example, a labeled antibody or lectin can be used to detect the presence of an antigen or carbohydrate, respectively, in, e.g., a tissue sample or on the surface of a cell, such as, e.g., a tumor-associated antigen. The presence of two or more detectable labels with the hairpin structure provides for increased sensitivity of detection, thereby allowing detection of low levels of the target molecule, such as, for example, the presence of a tumor-associated antigen on relatively few cells.

Linkers attaching the oligonucleotide and the biomolecule can be nucleotide or non-nucleotide linkers and can be essentially of any length and composition which do not interfere with formation of the hairpin structure or interaction of the biomolecule with the target molecule. The linker can be attached to the 5′ or 3′ end of the oligonucleotide hairpin structure or, alternatively, to an internal nucleotide. Nucleotide linkers can include a linker moiety for attachment of the biomolecule. For example, the oligonucleotide can include constituent bases having a polyamide backbone (see, e.g., Nielsen et al.), which can be conjugated to a peptide or polypeptide via a peptide linkage. (See, e.g., Awasthi and Nielsen, Methods Mol. Biol. 208:43-52, 2002; Awasthi and Nielsen, Comb. Chem. High Throughput Screen 5:253-9, 2002; Balasundaram et al., Bioorg. Med. Chem. 9:1115-21, 2001; Good et al., Nat. Biotechnol. 19:360-4, 2001.) Alternatively, other nucleotide derivatives in which the phosphodiester group has been replaced (e.g., phosphoramidite derivatives) can also be used. Examples of non-nucleotide linkers include polysaccharides, peptides, polypeptides, and sugar phosphate nucleotide backbones lacking a nucleotide nitrogenous base able to hydrogen bond to a nucleic acid. Additional examples of non-nucleotide linkers are provided in U.S. Pat. Nos. 5,585,481 and 5,696,251, both to Arnold, Jr. et al.

III. Detection of Target Nucleic Acids

In another aspect of the invention, methods are provided for detecting a target nucleic acid in a sample. The methods include the steps of (1) contacting the sample with a hybridization probe having one or more hairpin structures and having one or more detectably-labeled nucleotides within the hairpin structure(s); (2) incubating the sample and the probe under conditions to allow the probe to hybridize to a target nucleic acid within the sample; and (3) detecting the label on hybridized probe to detect the target nucleic acid.

Hybridization Probes

The probes used in the methods provided herein include hairpin-labeled hybridization probes as described in section II (Hairpin-labeled Probes), supra, including oligonucleotides having a labeled hairpin structure at one or both ends and dendrimers having one or more hairpin-labeled oligonucleotide branches. In addition, the probes can also include hairpin-labeled nucleic acid probes produced by primer extension of a hairpin-labeled oligonucleotide (e.g., by PCR or random priming) as described in section IV (Primer Extension of Hairpin-labeled Oligonucleotides), infra.

A population of probes can be homogeneous with respect to the target-binding region. Alternatively, a population of probes can comprise two or more probes with different target-binding regions. In certain embodiments, a large population of different probes is used. If more two or more different probes (i.e., probes with different target-binding regions) are used, the different probes can have different detectable labels.

In typical embodiments, the probes are used to detect nucleic acids in chromosomes, cells, tissues, cell-free mixtures of nucleic acids, and the like. The target-binding region of the probe can be, for example, degenerate, such as according to a partial amino acid sequence of a protein of interest. In other typical embodiments, the target binding region is predetermined (i.e., according to a predetermined nucleic acid such as, e.g., a target nucleic acid or a nucleic acid having substantial identity to a target). For example, the target-binding region can be designed to have substantial complementarity with nucleic acids associated with a particular tissue or cell or associated with a physiological condition of interest (e.g., with cellular functions such as, for example, proliferation, apoptosis, cell-cell interactions, secretion of proteins, and the like; with intracellular or extracellular signaling pathways; with a disease or disorder, including, e.g., cancer, immunological or inflammatory diseases, neurodegenerative diseases, diseases associated with viral infection and the like;). Target nucleic acids can include DNA or RNA, and can include, for example, nucleic acids associated with abnormal (e.g., increased or decreased) expression under a physiological condition (e.g., an aberrantly expressed RNA); with infectious agents (for example, viral nucleic acids such as, e.g., HIV or EBV nucleic acids); with mutations such as those linked to a disease or disorder (e.g., a mutant cellular gene or chromosome); and the like.

Samples

Samples used according to the methods provided herein include any sample suspected of containing a target nucleic acid. Samples can include, for example, those containing cells, organelles (e.g., nuclei), mitochondria and chloroplasts; chromosomes and fragments thereof; and viruses. Samples can be from any species including, e.g., mammals, fish, amphibians, avians, insects, protozoa, bacteria, eubacteria, and plants. Preferred mammals include primates (including, e.g., human), bovines, and rodents (e.g., mice, rats, rabbits, and guinea pigs). In certain methods, multiple samples (e.g., samples from more than one subject) can be pooled before analysis. Further, cells from a primary tissue can, for example, be analyzed directly for the presence of a target nucleic acid or propagated before analysis. In other embodiments, samples are from a homogeneous cell line. In yet other embodiments, samples are obtained from the tissue of a human subject.

In certain embodiments, isolated nucleic acids are immobilized onto a solid support (i.e., solid substrate or “matrix”). The solid support can comprise any material capable of binding DNA efficiently and uniformly while leaving surface-bound DNA both functional and accessible. Typically, suitable solid supports are chemically inert; allow high-density, stable binding of DNA, and, for use in detection of fluorescently labeled probes, have low intrinsic fluorescence while providing strong signal intensity with a broad dynamic range. Solid matrix substrates suitable for use in conjunction with the methods provided herein include, e.g., glass and membrane filters. For example, glass slides coated with amine or aldehyde surface chemistry are available from Corning Microarray Technology (CMT), Cel, and TeleChem International. Amine-coated glass slides can also be made in-house by treating glass slides with polylysine; details for the preparing polylysine slides are available on the Brown Laboratory Web Site. In addition, porous membrane materials (e.g., nitrocellulose, nylon, acrylamide, and the like) can also be used.

In certain embodiments, isolated nucleic acids from different tissues or cell types are spotted onto the solid support as an array, thereby allowing for analysis of tissue or cell-type specific analysis of target nucleic acids. For example, cDNAs corresponding to the mRNAs present in a set of tissue or cell samples can be quantitatively amplified using known methods (e.g., Quantitative PCR) and then spotted onto the solid substrate. The resulting spots are a quantitative representation of the relative distribution and expression of genes within the respective samples, and the array can be analyzed, e.g., for differential expression in the tissues or cells. The tissues or cells can be, for examples, different types of tissues or cells (e.g., kidney, spleen, thymus, or lung) or tissues or cells of the same type but exposed to different physiological conditions (e.g., presence of different pharmacological agents).

In yet other embodiments, tissue samples are spotted onto a solid support. The tissue samples can, for example, be spotted onto the solid support as an array of tissue samples. Such tissue arrays are particularly useful for, e.g., high-throughput expression studies, tissue-type specificity studies, and animal model analysis. Methods spotting tissue samples to construct tissue arrays are known in the art. For example, tissue arrays can be spotted on standard glass slides containing, e.g., 30-120 spotted tissue samples of 0.6-2 mm in diameter. The arrays can be made, e.g., with formalin-fixed or zinc-fixed paraffin embedded tissues. Tissues for constructing the arrays can, for example, be isolated normal animals, genetically modified animals (e.g., transgenics such as gene knockouts), or animals in an otherwise abnormal physiological condition such as, e.g., animal disease models and/or animals treated with different pharmacological agents.

Hybridization Conditions

Hybridization conditions suitable for use with detection probes described herein are known in the art. (See, e.g., Sambrook et al., supra; Ausubel et al., supra. See also Tijssen, Laboratory Techniques in Biochemistry and Molecular Biology, Vol. 24: Hybridization with Nucleic Acid Probes (Elsevier, N.Y. 1993). Hybridization is carried out under stringent conditions which allow formation of stable and specific binding of substantially complementary strands of nucleic acid and any washing conditions that remove non-specific binding of the probe. Generally, stringency occurs within a range from about 5° C. below the melting temperature (Tₘ) of the probe to about 20° C.-25° C. below the Tₘ. Stringency can be increased or decreased to specifically detect target nucleic acids having 100% complementarity or to also detect related nucleotide sequences having less than 100% complementarity. In certain methods, very stringent conditions are selected to be equal to the Tm for a particular probe. Factors such as the length and nature (DNA, RNA, base composition) of the sequence, nature of the target (DNA, RNA, base composition, presence in solution or immobilization) and the concentration of the salts and other components (e.g., the presence or absence of formamide, dextran sulfate and/or polyethylene glycol) are considered and the hybridization solution can be varied to generate conditions of either low, medium, or high stringency. Washing conditions typically range from room temperature to 60° C.

For example, high stringency conditions can include, e.g., 6×NaCl/sodium citrate (SSC) at about 45° C. for a hybridization step, followed by a wash of 2×SSC at 50° C.; or, alternatively, e.g., hybridization at 42° C. in 5×SSC, 20 mM NaPO4, pH 6.8, 50% formamide, followed by a wash of 0.2×SSC at 42° C. These conditions can be varied based on nucleotide base composition and length and circumstances of use, either empirically or based on formulas for determining such variation (see, e.g., Sambrook et al., supra; Ausubel et al., supra). Depending on base composition, source, and concentration of target nucleic acid, other conditions of stringency can be used, including, for example, low stringency conditions (e.g., 4-6×SSC/0.1-0.5% w/v SDS at 37-45° C. for 2-3 hours) or medium stringency conditions (e.g., 1-4×SSC/0.25-0.5% w/v SDS at 45° C. for 2-3 hours).

In general, there is a tradeoff between hybridization specificity (stringency) and signal intensity. In certain embodiments, the hybridized sample can be washed at successively higher stringency solutions and read between each wash. Analysis of the data sets thereby produced reveals a wash stringency above which the hybridization pattern is not appreciably altered and which provides adequate signal for the particular probes of interest.

Detection of Hybridized Probe

Following hybridization, the probe-target hybrid is detected using any methods suitable according to the type of detectable label present in the hairpin structure. As noted above in section II, labels can be either direct or indirect. Typical direct labels include fluorophores such as, e.g., fluorescein, rhodamine, Texas Red, phycoerythrin, Cy3, and Cy5. An indirect process utilizes a binding partner interaction for detection. The oligonucleotide probe is generally labeled with a molecule that has an affinity for a secondary agent. For example, biotin and haptens such as, e.g., digoxigenin (DIG), DNP, or flourescein are typical labels which can be detected via an interaction with streptavidin (in the case of biotin) or an antibody as the secondary agent. Following the hybridization step, the target-probe hybrid can be detected by using, e.g., directly labeled streptavidin or antibody. Alternatively, unlabeled secondary agents can be used with directly a labeled “tertiary” agent that specifically binds to the secondary agent (e.g., unlabeled anti-DIG antibody can be used, which can be detected with a labeled second antibody specific for the species and class of the primary antibody). The label for the secondary agent is typically a non-isotopic label, although radioisotopic labels can also be used. Typical non-isotopic labels include, e.g., enzymes and fluorophores, which can be conjugated to the secondary or tertiary agent. Enzymes commonly used in DNA diagnostics include, for example, horseradish peroxidase and alkaline phosphatase.

Detection of the probe label can be accomplished using any approach suitable for the particular label. For example, fluorophore labels can be detected using any suitable means known in the art for detecting the emission wavelength of the particular fluorophore used. Typical methods for detecting fluorescent signals include, e.g., spectrofluorimetry, confocal microscopy, and flow cytometry analysis. Fluorescent labels is generally preferred for detection of low levels of target nucleic acids because they provide a very strong signal with low background. Also, it is optically detectable at high resolution and sensitivity through a quick scanning procedure, and different hybridization probes having fluorophores with different emission wavelengths (e.g., fluorescein and rhodamine) can be used for a single sample.

In addition, with enzyme labels, detection can be, for example, by color or dye deposition (e.g., p-nitrophenyl phosphate or 5-bromo-4-chloro-3-indolyl phosphate/nitroblue tetrazolium for alkaline phosphatase and 3,3′-diaminobenzidine-NiCI.sub.2 for horseradish peroxidase), fluorescence (e.g., 4-methyl umbelliferyl phosphate for alkaline phosphatase) or chemiluminescence (e.g., the alkaline phosphatase dioxetane substrates LumiPhos 530 from Lumigen Inc., Detroit Mich. or AMPPD and CSPD from Tropix, Inc.). Chemiluminescent detection can be carried out with X-ray or Polaroid film or by using single photon counting luminometers. This is a typical detection format for alkaline phosphatase labeled probes.

In certain embodiments of the method, the detection assay is a solution phase assay. For example, target nucleic acids within cells in suspension (e.g., blood cells) can be hybridized to fluorescently-labeled probe in situ. In situ hybridization signals in individual cells can then be analyzed for the presence of target nucleic acids using a flow cytometer (e.g., a FACScan single laser flow cytometer).

IV. Primer Extension of Hairpin-labeled Oligonucleotides

In yet another aspect of the invention, primer-extended hairpin labeled probes and methods for primer extension of hairpin-labeled probes are provided. The methods for primer extension generally include contacting a target nucleic acid with an oligonucleotide hairpin-labeled probe as described in section II (Hairpin-labeled Probes), supra, wherein the hairpin structure is located 5′ to the target binding segment, under conditions that allow the target nucleic acid to serve as a template for primer extension of the hairpin-labeled probe.

Conditions suitable for primer extension are generally known in the art. (See, e.g., Sambrook et al., supra; Ausubel et al., supra.) The hairpin-labeled probe (the primer) is annealed, i.e., hybridized, to the target nucleic acid to form a primer-template complex. The primer-template complex is then exposed to a polymerization agent (e.g., a DNA polymerase), thermostable or otherwise, and to nucleotides or derivatives thereof in a suitable environment to permit the addition of one or more nucleotides or nucleotide derivatives to the 3′ end of the hairpin-labeled primer, thereby producing an extended primer complementary to the target nucleic acid. The primer extension reaction can employ an elevated temperature in order to denature double-stranded polynucleotides (such as in PCR primer extension reactions).

In some embodiments, the primer extension is performed in the presence of four unlabeled free nucleotides. In other embodiments, one or more of the free nucleotides are labeled. Labels can be direct or indirect, as described supra.

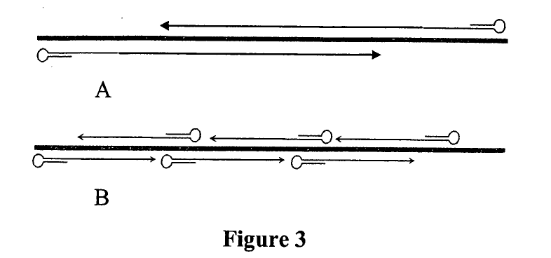

In certain embodiments, the target nucleic acid is randomly primed. (See, e.g., Feinberg and Vogelstein, Anal. Biochem. 137:266-7, 1984; Glick and Pasternak, Molecular Biotechnology: Principles and Applications of Recombinant DNA (ASM Press, 2d ed. 1998). See also FIG. 3B.) For these methods, the target-binding region of the hairpin-labeled primer is typically a random segment, such as, for example, a random segment of 3-10 nucleotides in length (e.g., a random hexamer (see, e.g., Feinberg and Vogelstein, supra)). For example, hairpin-labeled primers having random target-binding regions are added to a sample of denatured DNA, along with all four nucleotides and a DNA polymerase (e.g., the Klenow fragment). The use of random hexamers results in 4096 possible hexamer species, which stochastically bind to substantially complementary regions of the target nucleic acid according to the number of nucleotide positions in the random target-binding region. Once bound, the DNA polymerase incorporates complementary nucleotides to produce the extended primer.

In certain embodiments, the methods for primer extension further include contacting the target nucleic acid with a second primer that has a region substantially complementary to a segment of the extended primer under conditions that allow the target nucleic acid to serve as a template for amplification. (See FIG. 3A.) Conditions suitable for amplification of a target nucleic acid using a primer pair (i.e., a 5′ upstream primer and a 3′ downstream primer) are known in the art (e.g., PCR amplification methods). (See, e.g., Sambrook et al., supra; Ausubel et al.; supra; PCR Applications: Protocols for Functional Genomics (Innis et al. eds., Academic Press 1999); Glick and Pasternak, supra.) One or both primers can be hairpin-labeled; thus, one or both strands, respectively, of the amplification product can be hairpin-labeled.

Using the above methods, a hairpin-labeled probe having an extended target-binding region (herein a “primer-extended hairpin-labeled probe”) is produced. The primer-extended hairpin-labeled probes, including hairpin-labeled amplification products, and methods for using these probes in detection methods as described hereinabove, are also encompassed within the present invention.

V. Kits for Hybridization Detection Assays or Primer Extension

Also provided is a kit for utilizing the hybridization probes of the present invention in detection of target nucleic acids or, alternatively, for primer extension. Typically, the kit is compartmentalized for ease of use and contains at least one first container providing the oligonucleotide or dendrimer probe as described herein. Additional containers providing reagents for detecting the hairpin-labeled oligonucleotide or dendrimer probe and/or for primer extension of the hairpin-labeled oligonucleotide primer can also be included in the kit. Such additional containers can include any reagents or other elements recognized by the skilled artisan for use in hybridization detection assays or primer extension procedures in accordance with the methods provided herein. For example, in embodiments where the detectable label is indirect (e.g., biotin), at least one container providing a secondary agent for detection of the indirect label can be included (e.g., a container providing streptavidin labeled with a fluorophore). Also, kits for primer extension can also include at least one container providing a polymerization agent (e.g., DNA polymerase such as, for example, the Klenow fragment); at least one container providing an appropriate buffer (i.e., a buffer suitable for primer extension); at least one container providing labeled or unlabeled free nucleotides; and/or at least one container providing a second primer (e.g., a second hairpin-labeled oligonucleotide primer for PCR amplification of the target nucleic acid).

In particular embodiments, the kit for detection of a target nucleic acid is useful for diagnosis of a disease or disorder associated with a particular target nucleic acid. For example, target nucleic acids useful for diagnosis can include aberrantly expressed genes; nucleic acids associated with infectious agents such as, e.g., HIV or EBV; a mutant cellular gene or chromosome; an extra or missing gene or chromosome; and the like.

A further understanding of the present invention will be obtained by reference to the following description that sets forth illustrative embodiments.

### Example 1

### Detection of EBV EBER-1 RNA Using Hairpin-Labeled Oligonucleotides

Unless otherwise stated, all reagents are from Sigma-Aldrich Chemical, St. Louis, Mo. Equivalent reagents from sources other than those listed herein can also be used.

Cell Lines and Cell Culture Conditions

EBV-positive human RAJI cells and EBV-negative human RL and HL-60 cell lines were obtained from ATCC (Rockville, Md.) and cultured in RPMI 1640 medium supplemented with 2.0-4.5 g/l glucose, 2 mM L-glutamine and 10%-15% fetal bovine serum. Using PCR analysis, we confirmed that both RL and HL-60 lines are EBV-free. Raji cells contain approximately 50 copies of episomal EBV, and express EBER-1 RNA (Stevens et al. J. Clin. Microbiol. 37:2852-2857, 1999).

Cell Fixation

Cell fixation is required to maintain cellular structure, and retain target nucleic acids. Cell fixatives, such as HistoChoice-MB have been designed for molecular biology applications such as Fluorescent In Situ Hybridization (FISH), and are commercially available (Amresco Inc., Solon, Ohio). Cells are harvested by centrifugation (200×g), washed once in PBS, and resuspended in HistoChoice-MB fixative. Cells are either processed for hybridization immediately, or stored at 4° C.

Probes and Probe Labeling

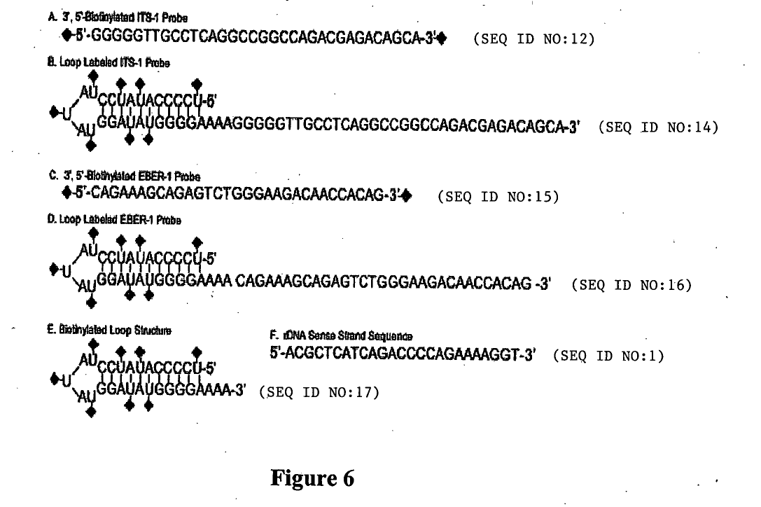

High copy numbers of EBER small nuclear RNAs are present in EBV latently infected cells (˜106 copies per cell, Clemens, Mol. Biol. Reports 17:81-92, 1993; Crouch et al., Cytometry 29:50-57, 1997), and represent a very desirable hybridization target. In related research, EBER-1 RNAs have successfully been detected in EBV-laden cells using a single oligonucleotide probe biotinylated at the 5′- and 3′-ends (FIG. 6C). Biotinylated 30-base random sequence (randomer) and rDNA sense strand (rDNA-sense) oligonucleotides (e.g., 5′-ACGCTCATCAGACCCCAGAAAAGGT-3′) serve as negative controls. The ITS-1 antisense pre-ribosomal RNA oligonucleotide probe previously discussed serves as the positive control. The oligonucleotide probes diagrammed in FIG. 6 are obtained from Oligos Etc. (Wilsonville, Oreg.).

Cytospin Slide Preparation and In Situ Hybridization

Fixed cells (2.5×10⁵ cells) are cytocentrifuged onto glass slides at 100×g for 5 min., using a Cytospin™ III cytocentrifuge (ThermoShandon Corp., Pittsburgh, Pa.). After air drying, cells are washed briefly in 2×SSC, then dehydrated in 70% and 95% ethanol. 3-5 μl of conventional or hairpin-labeled probe solution (25-250 nmol probe in 2×SSC/5% w/v polyethylene glycol 8000MW/10%-50% formamide/0.5% w/v bovine serum albumin [BSA]/5% v/v vanadyl ribonucleoside complex [VRC]/2000 nmol unlabeled random 30-mer oligonucleotide) is pipetted onto the cytocentrifuged cell spot, covered with Parafilm™, and incubated in a humidified chamber at 37° C. for 30 min. After hybridization, the Parafilm™ is removed, and the cell spot overlaid with 100 μl of Cy3-conjugated streptavidin (10 μg/ml in 4×SSC/0.5% w/v BSA/0.025% Triton X-100) at room temperature for 20 min. The slide is briefly washed in 4×SSC/0.5% Triton X-100, 2×SSC, and PBS. Total DNA is counterstained using DAPI (diamidinophenylindole; 2 μg/ml in PBS) for 30 sec, rinsed in PBS, and a glass coverslip mounted using anti-fade mounting media. Slides are analyzed using slide scanning cytometry, as described below.

Solution Phase In Situ Hybridization

Fixed cells (1.0×10⁶ cells) are pelleted by centrifugation at 400×g, and washed by resuspension in 2×SSC, and recovered by centrifugation. For hybridization, the cell pellet is resuspended in 50 μl of conventional or hairpin-labeled probe solution (25-250 nmol probe in 2×SSC/5% w/v polyethylene glycol 8000MW/10%-50% formamide/0.5% w/v bovine serum albumin [BSA]/5% v/v vanadyl ribonucleoside complex [VRC]/2000 nmol unlabeled random 30-mer oligonucleotide) and incubated in a shaking thermomixer at 37° C. for 1 hour. After incubation, cells are pelleted and washed as above. The cell pellet is resuspended in 250 μl of Cy3-conjugated streptavidin (10 μg/ml in 4×SSC/0.5% w/v BSA/0.025% Triton X-100) and incubated at room temperature for 20 min. Cells are washed twice in 4×SSC/0.5% Triton X-100, 2×SSC, then resuspended in PBS. Samples are analyzed by flow cytometry as detailed below.

Slide Analysis, Image Scanning Cytometry and Flow Cytometry

Digital imaging is performed using IPLab Spectrum software (Scanalytics, Inc., Fairfax, Va.) on a Nikon E600 microscope equipped for epifluorescence. For flow cytometry analysis, samples are analyzed using a FACScan single laser flow cytometer, using CellQuest acquisition and analysis software (Becton Dickinson Immunocytometry Systems, San Jose, Calif.; ver. 3.2.1). Signal for Cy3-labeled samples are acquired using FL3 channel, and dot plot analysis of positive and negative samples used to determine result gating.

In Situ Detection of EBV EBER-1 RNA

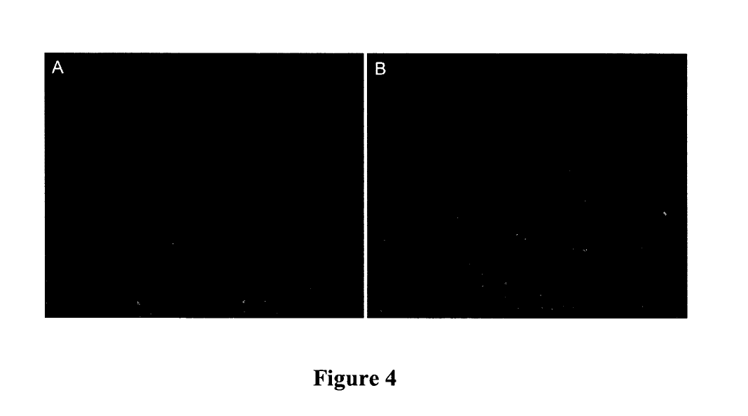

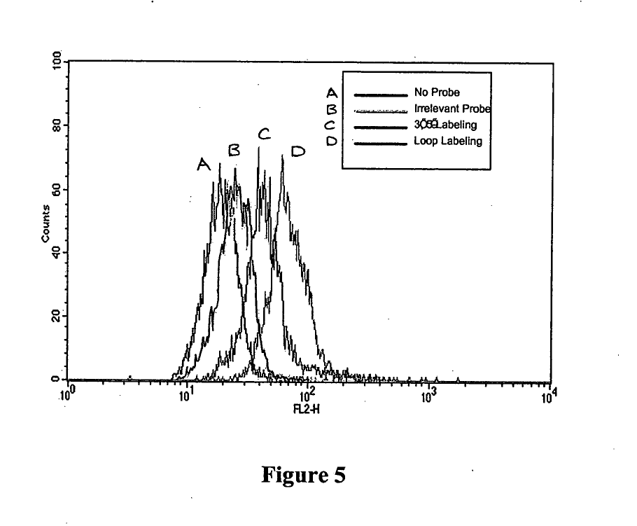

As shown, EBV EBER-1 RNA was successfully detected using both flow cytometry solution phase in situ hybridization and slide based hybridization procedures (FIGS. 4, 5). The EBER-1 RNA is a preferred hybridization target for detecting EBV, due to their abundance; up to 1×10⁷ copies/cell, in latently infected cells (Clemens, supra; Crouch et al., supra; Stowe et al., J Vir. Meth. 75:83-91, 1998), and in virtually all infected B-cells in lymphoproliferative disease (Baumforth et al., Mol. Pathol. 52:307-322, 1999). As a result, it is a suitable model system for evaluating new probe designs, as well as a bona fide target of clinical significance. Using flow cytometry, EBV-positive cells in a background of negative cells were detected, with clear discrimination between positive and negative cells (FIG. 4). Conventional slide-based FISH, in conjunction with image scanning cytometry, also was used to detect EBV-positive cells (FIG. 5). Due to the high target copy number, unambiguous detection of positive cells was able to be performed (FIG. 5). Rapid slide scanning is a key requirement for analyzing large cell numbers of cells. A substantial increase in scanning rate over that used here would be attainable if shorter image acquisition times can be used. This is accomplished by increasing the hybridization signal intensity using hairpin-labeled oligonucleotide probes.

In Situ Hybridization Using Hairpin-Labeled Oligonucleotide Probes

To assess whether hairpin-labeled oligonucleotide probes can be used successfully for in situ hybridization detection of RNA, a loop labeled probe recognizing the Internal Transcribed Sequence-1 (ITS-1) region of ribosomal RNA was synthesized. For comparison purposes, the same sequence was labeled using conventional 3′,5′-biotinylation. Following hybridization under identical conditions, using both slide-based and solution phase hybridization methods, followed by detection using Cy3-conjugated streptavidin, hybridization signal intensity and specificity was assessed using conventional epifluorescence microscopy and flow cytometry. As shown, hybridization signal specificity was identical for both types of probes, and was restricted to nucleoli, the demonstrated site of ribosomal gene transcription (FIGS. 4A, 4B). Hybridization signal was totally removed by RNAse treatment prior to hybridization, and by hybridization in the presence of a large molar excess of non-labeled identical sequence oligonucleotide, both key methods for confirming hybridization specificity (data not shown). Hybridization signal intensity was noticeably brighter for the hairpin-labeled probe (FIG. 4B), compared to the conventional labeled probe (FIG. 4A). Using flow cytometry analysis of solution phase hybridizations, it was clearly demonstrated that hybridization performed using the hairpin-labeled probe exhibited increased fluorescence intensity (FIG. 5; histogram D) compared to that using the conventional probe (FIG. 5; histogram C).

### Example 2

### Detection of HIV RNA Using Hairpin-Labeled Oligonucleotides

Materials and Methods

Unless otherwise noted, all reagents are from Sigma-Aldrich Chemical, St. Louis, Mo. Equivalent reagents from sources other than those listed herein can also be used.

Cells

OM10.1 cells, latently infected with HIV-1, are used to detect HIV-RNA expressed in cells. HL-60, human promyelocytic leukemia cells, used to generate OM10.1 cells, are used as control cells. Expression of HIV RNA in OM10.1 cells is induced with 20 U/ml of TNFα for 16 hours. Peripheral blood mononuclear cells are isolated from human blood collected in either K3EDTA or citrate Vacutainer (Becton and Dickinson) tubes using Ficoll-Paque Plus (Amersham Biosciences) density gradient. Blood from HIV infected humans is obtained from Mass. General Hospital (Boston, Mass.) or Research Sample Bank (Pompano Beach, Fla.). If fresh blood is not used, complete removal of granulocytes is aided with use of RosetteSep™ (StemCell Technologies) method, which relies on crosslinking granulocytes with red blood cells applying tetrameric complexes. Because yields of PBMCs from density gradient separations are in the range of only 40-60%, HIV infected cells can be preferentially lost during this procedure. Therefore, use of whole blood after selective lysis of red blood cells is also examined. To 1 ml of whole blood, 100 μl of fluorescein labeled antibody against CD4 or CD14 is added and, after vortexing, the mixture is kept at room temperature for 15 min. Then, 1 ml of 4% paraformaldehyde in PBS is added and, after 15 min, 10 ml of PBS containing 0.1 ml of Ribonucleoside Vanadyl Complex (RVC, New England Biolabs, Beverly, Mass.) is added to the blood. After mixing, blood cells are pelleted by 5 min centrifugation at 300×g. Supernatant is discarded and cells are washed twice with 10 ml PBS. After the washing steps, 2 ml PBS containing 0.1% saponin and 20 μl RVC is added to pelleted blood cells. White blood cells (WBC) are permeabilized and red blood cells (RBC) are lysed during this 10 min step. Permeabilized WBC are separated from lysed RBC by centrifugation and two washing steps using PBS containing RVC. Pelleted cells are used immediately for in situ hybridization.

Probes

For detecting HIV RNA, a cocktail of the following hairpin biotin-labeled oligonucleotide probes covering gag and pol region of HIV genome is used:

HIV-1 5′ ctc tgg tct gct ctg aag aaa tgg tg 3′
HIV-2 5′ ggt cgt tgc caa aga gtg atc tga g 3′
HIV-3 5′ cat ttc ttc tag tgt agc tgc tgg tcc 3′
HIV-4 5′ctg cca gtt cta gct ctg ctt ctt c 3′
HIV-5 5′ cta gct gcc cca tet aca tag aac g 3′
HIV-6 5′ ctg cta tgt cac ttc ccc ttg gtt ctc 3′
HIV-7 5′ gct ccc tgc ttg ccc ata cta tat g 3′
HIV-8 5′ cta ata gag ctt cct tta gtt gcc ccc 3′
HIV-9 5′ gca tca ccc aca toc agt act gtt ac 3′

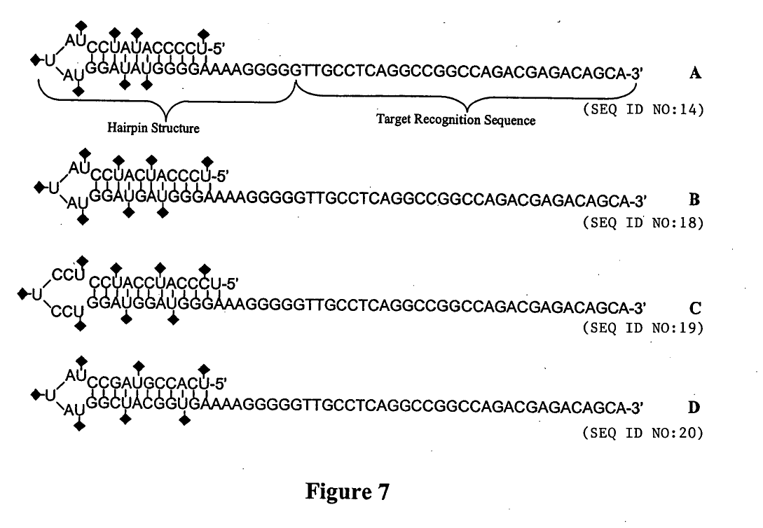

Four different hairpin structures linked with HIV-1 probe are used, as depicted in FIG. 7.

In Situ Hybridization in Solution

Cells are fixed for 30 min with 4% paraformaldehyde and quantified using a hemocytometer. 2×10⁶ cells are used for the analysis. Cells are permeabilized in PBS containing 0.1% saponin and 100 U/ml SUPERase (RNase inhibitor from AMBION, Austin, Tex.) and hybridized in a solution containing a cocktail of probes at 0.2 μg/ml in 2×SSC, 10 mM MES pH 6.7, 1% bovine serum albumin, 25% formamide, 1 mg/ml both calf thymus DNA and tRNA and 0.1% pluronic acid. After hybridizing for 1 hour at 48° C., cells are pelleted and washed once in 2×SSC, 25% formamide, 0.1% pluronic acid, once in 4×SSC, 0.5% bovine serum albumin and stained 15 min in Streptavidin-Phycoerythrin (2 μg/ml, Molecular Probes, Eugene, Oreg.) dispersed in the latter solution. After washing, cellular DNA is stained with DRAQ5 (20 μM).

Immunotyping

During analysis of clinical blood samples, peripheral blood cells are immunotyped with fluorescein labeled antibodies against CD4 or CD14 (Beckman-Coulter, Miami, Fla.) in PBS containing 0.5% bovine serum albumin and 0.1% sodium azide. After staining for 15 min, cells are washed with this solution and fixed with 4% paraformaldehyde. Cells are stored for at least 15 min in 0.15 M ammonium sulfate containing 0.1% Pluronic acid (solution blocks free aldehyde groups) until in situ hybridization is performed.

Flow Cytometry Analysis

In situ hybridization signals in individual cells are analyzed using a FACScan single laser flow cytometer equipped with CellQuest acquisition and analysis software (Becton Dickinson Immunocytometry Systems, San Jose, Calif.). CD4 or CD14 signals and HIV RNA are collected in FL1 and FL2 channels, respectively, after setting the appropriate compensation. DNA signals are collected in the FL3 channel. We have determined that compensation between FL2 (phycoerythrin) and FL3 (DRAQ5) channels is not necessary. Using Poisson statistics, for a confidence level of 10%, 100 positive events should be acquired. Assuming that the subpopulation of HIV-RNA containing cells represents ≧0.02%, analysis of approximately 1×10⁶ events is required for statistically meaningful analyses.

The previous examples are provided to illustrate but not to limit the scope of the claimed invention. Other variants of the inventions will be readily apparent to those of ordinary skill in the art and encompassed by the appended claims. All publications, patents, patent applications, and other references cited herein are hereby incorporated by reference.

1. A labeled oligonucleotide comprising:
a single-stranded target-binding segment substantially complementary to a target nucleic acid; and
a hairpin structure comprising a stem region and a loop region, wherein a plurality of nucleotides within the hairpin structure have a detectable label.

2. The labeled oligonucleotide of claim 1, further comprising a linker between the target-binding segment and the hairpin structure.

3. The labeled oligonucleotide of claim 1, wherein at least one nucleotide in the loop region has the detectable label.

4. The labeled oligonucleotide of claim 1, wherein at least one nucleotide in the stem region has the detectable label.

5. The labeled oligonucleotide of claim 4, wherein at least one nucleotide in the loop region has the detectable label.

6. The labeled oligonucleotide of claim 1, wherein the plurality of nucleotides having the detectable label is at least five.

7. The labeled oligonucleotide of claim 1, wherein the plurality of nucleotides having the detectable label is nine.

8. The labeled oligonucleotide of claim 1, wherein the labeled oligonucleotide has up to 60 nucleotides.

9. The labeled oligonucleotide of claim 1, wherein the labeled oligonucleotide has up to 100 nucleotides.

10. The labeled oligonucleotide of claim 1, wherein the labeled oligonucleotide has up to 150 nucleotides.

11. The labeled oligonucleotide of claim 1, wherein the loop region has 3-10 nucleotides.

12. The labeled oligonucleotide of claim 1, wherein the stem region has 16-40 nucleotides.

13. The labeled oligonucleotide of claim 1, wherein at least two nucleotides having the detectable label are adjacent.

14. The labeled oligonucleotide of claim 1, wherein the nucleotides having the detectable label are spaced at least two nucleotides apart.

15. The labeled oligonucleotide of claim 1, wherein the nucleotides having the detectable label are spaced 2-6 nucleotides apart.

16. The labeled oligonucleotide of claim 1, wherein the target-binding segment is a predetermined segment.

17. The labeled oligonucleotide of claim 16, wherein the target nucleic acid is a viral nucleic acid.

18. The labeled oligonucleotide of claim 17, wherein the viral nucleic acid is an HIV or EBV nucleic acid.

19. The labeled oligonucleotide of claim 1, wherein the target-binding segment is a random segment.

20. The labeled oligonucleotide of claim 1, wherein the target-binding segment is a degenerate segment.

21. The labeled oligonucleotide of claim 1, wherein the detectable label is an indirect label.

22. The labeled oligonucleotide of claim 21, wherein the indirect label is biotin.

23. The labeled oligonucleotide of claim 21, wherein the indirect label is a hapten.

24. The labeled oligonucleotide of claim 23, wherein the hapten is selected from the group consisting of digoxigenin, dinitrophenol (DNP), biotin, and fluorescein.

25. The labeled oligonucleotide of claim 1, wherein the detectable label is a direct label.

26. The labeled oligonucleotide of claim 25, wherein the direct label is a fluorophore.

27. The labeled oligonucleotide of claim 26, wherein the fluorophore is selected from the group consisting of fluorescein, rhodamine, Texas Red, phycoerythrin, Cy3, and Cy5.

28. The labeled oligonucleotide of claim 1, further comprising:
a second hairpin structure comprising a second stem region and a second loop region, wherein at least one nucleotide within the second hairpin structure has the detectable label; and
wherein the hairpin structures are linked to opposite ends of the target-binding segment.

29. A labeled oligonucleotide comprising:
a single-stranded target-binding segment substantially complementary to a target nucleic acid;
a first hairpin structure comprising a first stem region and a first loop region; and
a second hairpin structure comprising a second stem region and a second loop region;
wherein at least one nucleotide within the first hairpin structure and at least one nucleotide within the second hairpin structure have a detectable label; and
wherein the hairpin structures are linked to opposite ends of the target-binding segment.

30. A dendrimer probe comprising:
a plurality of labeled oligonucleotides according to claim 1; and a branching molecule linking the oligonucleotides.

31. A labeled biomolecule comprising:
an oligonucleotide that forms a hairpin structure comprising a stem region and a loop region, wherein a plurality of nucleotides within the hairpin structure have a detectable label; and
a linker attaching the oligonucleotide and the biomolecule.

32. A method for detecting a target nucleic acid in a sample, the method comprising:
1) contacting the sample with an oligonucleotide probe, the oligonucleotide probe comprising
a) a single-stranded target-binding segment substantially complementary to the target nucleic acid; and
b) a hairpin structure comprising a stem region and a loop region, wherein a plurality of nucleotides within the hairpin structure have a detectable label;
2) incubating the sample and the oligonucleotide probe under conditions sufficient to allow the target-binding segment to hybridize to the target nucleic acid; and
3) detecting the label on hybridized oligonucleotide probe to detect the target nucleic acid.

33. The method of claim 32, further comprising removing non-hybridized oligonucleotide probe before detecting the label.

34. The method of claim 32, wherein the oligonucleotide probe further comprises a linker between the target-binding segment and the hairpin structure.

35. The method of claim 32, wherein at least one nucleotide in the loop region has the detectable label.

36. The method of claim 32, wherein at least one nucleotide in the stem region has the detectable label.

37. The method of claim 36, wherein at least one nucleotide in the loop region has the detectable label.

38. The method of claim 32, wherein the plurality of nucleotides having the detectable label is at least five.

39. The method of claim 32, wherein the plurality of nucleotides having the detectable label is nine.

40. The method of claim 32, wherein the oligonucleotide probe has up to 60 nucleotides.

41. The method of claim 32, wherein the oligonucleotide probe has up to 100 nucleotides.

42. The method of claim 32, wherein the oligonucleotide probe has up to 150 nucleotides.

43. The method of claim 32, wherein the loop region has 3-10 nucleotides.

44. The method of claim 32, wherein the stem region has 16-40 nucleotides.

45. The method of claim 32, wherein the nucleotides having the detectable label are spaced at least two nucleotides apart.

46. The method of claim 32, wherein at least two nucleotides having the detectable label are adjacent.

47. The method of claim 32, wherein the nucleotides having the detectable label are spaced 2-6 nucleotides apart.

48. The method of claim 32, wherein the target-binding segment is a predetermined segment.

49. The method of claim 48, wherein the predetermined nucleic acid is a viral nucleic acid.

50. The method of claim 49, wherein the viral nucleic acid an HIV or EBV nucleic acid.

51. The method of claim 32, wherein the target-binding segment is a degenerate segment.

52. The method of claim 32, wherein the detectable label is an indirect label and the detection comprises contacting the indirect label with a secondary label.

53. The method of claim 52, wherein the indirect label is biotin.

54. The method of claim 53, wherein the secondary label is labeled streptavidin.

55. The method of claim 52, wherein the indirect label is a hapten.

56. The method of claim 55, wherein the hapten is selected from the group consisting of digoxigenin, dinitrophenol (DNP), biotin, and fluorescein.

57. The method of claim 55, wherein the secondary label is a labeled anti-hapten antibody.

58. The method of claim 32, wherein the detectable label is a direct label.

59. The method of claim 58, wherein the direct label is a fluorophore.

60. The method of claim 59, wherein the fluorophore is selected from the group consisting of fluorescein, rhodamine, Texas Red, phycoerythrin, Cy3, and Cy5.

61. The method of claim 32, wherein the oligonucleotide probe further comprises a second hairpin structure comprising a second stem region and a second loop region, wherein at least one nucleotide within the second hairpin structure has the detectable label; and wherein the hairpin structures are linked to opposite ends of the target-binding segment.

62. The method of claim 32, wherein the target nucleic acid is immobilized on a solid substrate.

63. The method of claim 32, wherein the target nucleic acid is within a cell or tissue sample, and the labeled oligonucleotide hybridizes to the target nucleic acid in situ.

64. The method of claim 63, wherein the detection of the label on hybridized oligonucleotide probe comprises a solution phase assay.

65. The method of claim 64, wherein the solution phase assay comprises flow cytometry.

66. A method for detecting a target nucleic acid in a sample, the method comprising:
1) contacting the sample with an oligonucleotide probe, the oligonucleotide probe comprising
a) a single-stranded target-binding segment substantially complementary to the target nucleic acid;
b) a first hairpin structure comprising a first stem region and a first loop region;
c) a second hairpin structure comprising a second stem region and a second loop region;
wherein at least one nucleotide within the first hairpin structure and at least one nucleotide within the second hairpin structure have a detectable label; and
wherein the hairpin structures are linked to opposite ends of the target-binding segment;
2) incubating the sample and the oligonucleotide probe under conditions sufficient to allow the target binding segment to hybridize to the target nucleic acid; and
3) detecting the label on hybridized oligonucleotide probe to detect the target nucleic acid.

67. A method for detecting a target nucleic acid in a sample, the method comprising:
1) contacting the sample with a dendrimer probe according to claim 30;
2) incubating the sample and the dendrimer probe under conditions sufficient to allow the target-binding segment to hybridize to the target nucleic acid; and
3) detecting the label on hybridized dendrimer probe to detect the target nucleic acid.

68. A method for conducting primer extension comprising:
contacting a target nucleic acid with an oligonucleotide primer according to claim 1, wherein the hairpin structure is located 5′ to the target-binding segment, under conditions whereby the target nucleic acid serves as a template for extension from the primer to produce an extended primer.

69. The method of claim 68, further comprising contacting the target nucleic acid with a second primer, said second primer comprising a priming segment substantially complementary to the extended primer, under conditions whereby the target nucleic acid serves as a template for amplification from the oligonucleotide primer and the second primer to produce an amplification product.

70. The method of claim 68, wherein the amplification is performed in the presence of unlabeled free nucleotides.

71. The method of claim 68, wherein the target-binding segment is random.

72. The method of claim 71, wherein the random target-binding segment consists of 3-10 nucleotides.

73. The method of claim 68, wherein the second oligonucleotide primer further comprises, located 5′ to the priming segment, a second hairpin structure comprising a second stem region and a second loop region, wherein at least one nucleotide within the second hairpin structure has the detectable label.

74. A method for producing a nucleic acid amplification product, the method comprising:
contacting a target nucleic acid with
a) a first oligonucleotide primer comprising
i) a single-stranded target-binding segment substantially complementary to a target nucleic acid; and
ii) located 5′ to the target-binding segment, a first hairpin structure comprising a first stem region and a first loop region; wherein at least one nucleotide within the first hairpin structure has a detectable label;
said contacting comprising conditions whereby the target nucleic acid serves as a template for extension from the first primer to produce an extended primer; and
b) a second oligonucleotide primer comprising
i) a priming segment substantially complementary to the extended primer; and
ii) located 5′ to the priming segment, a second hairpin structure comprising a second stem region and a second loop region, wherein at least one nucleotide within the second hairpin structure has the detectable label;
said contacting further comprising conditions whereby the target nucleic acid serves as a template for amplification from the first and second oligonucleotide primers to produce an amplification product.

75. A kit for detection of a target nucleic acid, comprising:
at least one first container providing either the labeled oligonucleotide according to claim 1 or the dendrimer probe according to claim 30.

76. The kit according to claim 75, wherein the detectable label is an indirect label and further comprising at least one second container providing a secondary agent for detecting the indirect label.

77. A kit for primer extension of an oligonucleotide primer, comprising:
at least one first container providing a labeled oligonucleotide primer according to claim 1, wherein the hairpin structure is located 5′ to the target-binding segment.

78. The kit according to claim 77, further comprising at least one second container providing a second primer, said second primer comprising a priming segment substantially complementary to an extended primer produced under conditions whereby the target nucleic acid serves as a template for extension from the labeled oligonucleotide primer.

79. The kit according to claim 78, further comprising at least one third container providing labeled or unlabeled free nucleotides, at least one fourth container providing a polymerization agent, and at least one fifth container providing a buffer suitable for primer extension.


SEQUENCE LISTING (20 sequences)

SEQ ID NO: 1 - DNA, 25 bp, Artificial Sequence
  rDNA sense strand sequence
acgctcatcagaccccagaaaaggt

SEQ ID NO: 2 - DNA, 26 bp, Artificial Sequence
  oligonucleotide probe
ctctggtctgctctgaagaaatggtg

SEQ ID NO: 3 - DNA, 25 bp, Artificial Sequence
  oligonucleotide probe
ggtcgttgccaaagagtgatctgag

SEQ ID NO: 4 - DNA, 27 bp, Artificial Sequence
  HIV-1 oligonucleotide probe
catttcttctagtgtagctgctggtcc

SEQ ID NO: 5 - DNA, 25 bp, Artificial Sequence
  HIV-1 oligonucleotide probe
ctgccagttctagctctgcttcttc

SEQ ID NO: 6 - DNA, 25 bp, Artificial Sequence
  HIV-1 oligonucleotide probe
ctagctgccccatctacatagaacg

SEQ ID NO: 7 - DNA, 27 bp, Artificial Sequence
  HIV-1 oligonucleotide probe
ctgctatgtcacttccccttggttctc

SEQ ID NO: 8 - DNA, 25 bp, Artificial Sequence
  HIV-1 oligonucleotide probe
gctccctgcttgcccatactatatg

SEQ ID NO: 9 - DNA, 27 bp, Artificial Sequence
  HIV-1 oligonucleotide probe
ctaatagagcttcctttagttgccccc

SEQ ID NO: 10 - DNA, 26 bp, Artificial Sequence
  HIV-1 oligonucleotide probe
gcatcacccacatccagtactgttac

SEQ ID NO: 11 - DNA, 42 bp, Artificial Sequence
  HIV-1 oligonucleotide probe
gggggttgcctcaggccggccagacgagacagcauuuuuuuu

SEQ ID NO: 12 - DNA, 34 bp, Artificial Sequence
  ITS-1oligonucleotide probe
gggggttgcctcaggccggccagacgagacagca

SEQ ID NO: 13 - DNA, 34 bp, Artificial Sequence
  oligonucleotide probe
ggggguugccucaggccggccagacgagacagca

SEQ ID NO: 14 - DNA, 64 bp, Artificial Sequence
  ITS-1 oligonucleotide probe
uccccauauccuauauggauauggggaaaagggggttgcctcaggccggccagacgagacagca

SEQ ID NO: 15 - DNA, 31 bp, Artificial Sequence
  EBER-1 oligonucleotide probe
cagaaagcagagtctgggaagacaaccacag

SEQ ID NO: 16 - DNA, 61 bp, Artificial Sequence
  EBER-1 oligonucleotide probe
uccccauauccuauauggauauggggaaaacagaaagcagagtctgggaagacaaccacag

SEQ ID NO: 17 - DNA, 30 bp, Artificial Sequence
  hairpin labeling stem and loop sequence
uccccauauccuauauggauauggggaaaa

SEQ ID NO: 18 - DNA, 64 bp, Artificial Sequence
  oligonucleotide probe
ucccaucauccuauauggaugaugggaaaagggggttgcctcaggccggccagacgagacagca

SEQ ID NO: 19 - DNA, 67 bp, Artificial Sequence
  oligonucleotide probe
ucccauccauccuccuccuggauggaugggaaagggggttgcctcaggccggccagacgagacagca

SEQ ID NO: 20 - DNA, 64 bp, Artificial Sequence
  oligonucleotide probe
ucaccguagccuauauggcuacggugaaaagggggttgcctcaggccggccagacgagacagca